(19) United States
(12) Patent Application Publication
(10) Pub. No.: US 2009/0306355 A1
(43) Pub. Date: Dec. 10, 2009

(54) Preparation of 3,3'-diamino-4,4'-azoxyfurazan, 3,3'-4,4'-azofurazan, and pressed articles

(75) Inventors: David E. Chavez, Rancho de Taos, NM (US); Elizabeth G. Francois, Jemez Springs, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(73) Assignee: The Regents of the University of California

(21) Appl. No.: 12/156,904

(22) Filed: Jun. 4, 2008

(51) Int. Cl. C07C 291/08; C07C 245/02

(52) U.S. Cl. 534/572; 534/733

(57) ABSTRACT

A method for preparing essentially pure 3,3′-diamino-4,4′-azoxyfurazan (“DAAF”) that involves reacting an aqueous solution that includes 3,4-diaminofurazan (“DAF”) with OXONE™ (i.e. 2KHSO₅.KHSO₄.K₂SO₄) in the presence of a chemical buffer or a material that produces a chemical buffer during the reaction between DAF and the OXONE™. The essentially pure product particles can be used without further purification in preparing pressed articles of that consist of essentially pure DAAF. When sodium hypochlorite is used instead of OXONE, the product is 3,3′-diamino-4,4′-azofurazan (“DAAzF”).

### STATEMENT REGARDING FEDERAL RIGHTS

This invention was made with government support under Contract No. DE-AC52-06NA25396 awarded by the U.S. Department of Energy awarded by the U.S. Department of Energy. The government has certain rights in the invention.

### FIELD OF THE INVENTION

The present invention relates generally to the preparation of essentially pure 3,3′-diamino-4,4′-azoxyfurazan (“DMFA”) from 3,4-diaminofurazan (“DAF”), and to the preparation of pressed articles of essentially pure DAAF, and also to the preparation of 3,3′-diamino-4,4′-azofurazan (“DAAzF”).

### BACKGROUND OF THE INVENTION

The synthesis of 3,4-diaminofurazan (“DAF”) was first reported by Coburn in J. Heterocyclic Chem., vol. 5, (1968), pages 83-87. Since then, a large body of work has accumulated on the oxidation of DAF, especially by Russian scientists, e.g., Solodyuk et al., in Zh. Org. Khim., vol. 17(4), pp. 756-759 (1981). They used a variety of peroxide reagents on DAF to prepare 3,3′-diamino-4,4′-azoxyfurazan (“DAAF”), 3,3′-diamino-4,4′-azofurazan (“DAAzF”), and 3-amino-4-nitrofurazan, usually as impure mixtures.

Later, Hiskey et al. in U.S. Pat. No. 6,358,339, incorporated by reference herein, found that DAAF and DAAzF are insensitive high explosive materials, where “insensitive” means that the material has a drop height of greater than 320 cm as measured by using a 2.5 kg falling weight (Type 12). Hiskey et al. found that DAAF had a drop height of greater than 320 cm (2.5 Kg, Type 12) and elicited no response to spark (>0.36 J) or friction (>36 kg, BAM). Hiskey et al. prepared the DAAF according to the earlier Russian procedures, pressed low-density pellets of the DAAF that resulted, and found that this DAAF could be pressed neat but high-density pellets required a formulation with 5 volume percent of latex Kel-F 800 resin (a chlorotrifluoroethylene/vinylidene fluoride copolymer, available from 3M Company). The addition of Kel-F made it possible to press pieces up to a density of 1.70 g/cm³ (97% of theoretical maximum density). Hiskey et al. also examined the explosive performance properties of this DAAF using the known poly-ρ test, which is a test that determines detonation velocity as a function of density. Hiskey performed this test at two diameters, 0.5 inches and 0.25 inches, and showed that the detonation velocity for the DAAF/Kel-F formulations were relatively independent of diameter. The detonation velocity was determined to be 8.0 kilometers per second (km/s) at a density of 1.69 g/cm³. This data was further verified by an unconfined rate stick of pellets at a density of 1.69 g/cm³ and 3 mm in diameter. A complete detonation was achieved as evidenced by a witness plate. Although an accurate detonation velocity could not be obtained, the detonation pressure (P_{CJ}) was estimated to be 299 kbar from a 0.5-inch diameter plate dent at a density of 1.69 g/cm³. Hiskey also studies the shock sensitivity by performing wedge tests of the DAAF/5% Kel-F 800 resin formulation pressed to 1.705 g/cm³. Results from the wedge testing showed that the shock sensitivity of DAAF/Kel-F was like that for HMX. Hiskey et al. also characterized the explosive energy by performing a standard 1-inch cylinder test on DAAF that was neat-pressed to a density of 1.691 g/cm³. Hiskey et al. in U.S. Pat. No. 6,358,339 also reported an improved synthesis of DAAzF.

It is an object of this invention to provide an improved process for preparing essentially pure 3,3′-diamino-4,4′-azoxyfurazan (“DAAF”).

It is another object of this invention to provide an improved process for preparing a pressed article, a pellet for example, of essentially pure DAAF.

### SUMMARY OF THE INVENTION

The present invention includes a simple method for preparing essentially pure 3,3′-diaminoazoxy furazan. The method involves reacting an aqueous solution having 3,4-diaminofurazan (“DAF”) with a material of the formula 2KHSO₅.KHSO₄.K₂SO₄ in the presence of a chemical buffer or a material that produces a chemical buffer during the reaction of the DAF with the material of the formula 2 KHSO₅.KHSO₄.K₂SO₄, wherein DAAF precipitates from the aqueous solution.

The invention also includes a method of preparing a pressed article that consists essentially of 3,3′-diamino-4,4′-azoxyfurazan (“DAAF”) by reacting an aqueous solution comprising 3,4-diaminofurazan (“DAF”) with a material of the formula 2 KHSO₅.KHSO₄.K₂SO₄ at a pH in a range of from about 6.0 to about 8.0, thereby producing particles of essentially pure DAAF particles, and thereafter without any intermediate recrystallization, pressing the essentially pure DAAF particles into a pressed article.

The invention also includes a method of preparing 3,3′-diamino-4,4′-azofurazan (“DAAzF”). This method includes, comprising reacting an aqueous solution comprising 3,4-diaminofurazan (“DAF”) with sodium hypochlorite.

### DETAILED DESCRIPTION

The present invention is a simple method for synthesizing essentially pure 3,3′-diamino-4,4′-azoxyfurazan (“DAAF”) from 3,4′-diaminofurazan (“DAF”). The method results in essentially pure DAAF without any hazardous coproducts. The synthesis is performed using OXONE™ and a chemical buffer. The chemical buffer can be added prior to combining the DAF and OXONE™, or a material can be added that produces the chemical buffer as DAF reacts with OXONE™. In some runs, the pH was measured and appears to vary in at least one case from a pH of about 6.0 to a pH of about 8.0 while still producing essentially pure DMF. OXONE™ is known in the art to have the formula 2KHSO₅.KHSO₄.K₂SO₄. The reaction is shown below.

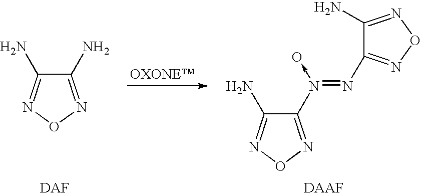

An aspect of the invention is that the process results in DAAF that is essentially pure and does not require any additional recrystallization or purification. According to the present process, DAAF precipitates out of the reaction mixture and after it is filtered from the reaction mixture, it is merely washed and dried. As a direct result of forming essentially pure DAAF directly from the reaction mixture, articles of DAAF can be pressed directly from the product without any intermediate purification steps.

Another aspect of the invention is related to the density and measured detonation velocity of pressed articles formed from the essentially pure DAAF prepared according to the invention. Generally, the usefulness of a pressed article of DAAF improves as the density approaches the theoretical maximum density (“TMD”) of DAAF, which is 1.747 grams per cubic centimeter. Pressed articles of DAAF have been reported with high densities. However, these pressed articles are not of essentially pure DAAF. Although articles of essentially pure DAAF can be pressed from recrystallized DAAF, the densities are significantly lower than TMD value because the particle size of recrystallized DAAF is quite small. Pressed articles of essentially pure DMF with higher densities have been prepared and their detonation velocities have been measured.

The invention is also concerned with a simple process for preparing 3,3′-diamino-4,4′-azofurazan (“DAAzF”). This process involves reacting DAF with sodium hypochlorite. Household bleach could be used as a reagent. The method is improved when the bleach is buffered.

The following EXAMPLES illustrate the preparation of DAAF and of pressed articles of DMF. The pH and temperature were measured using standard measuring techniques. Differential Scanning Calorimetry (“DSC”) of the DAAF product was used to determine purity.

### Example 1

An aqueous solution of 3,4-diaminofurazan (“DAF”) (1.02 grams, 10.2 millimoles (“mmol”)) and sodium bicarbonate (2.52 grams, 30 mmol) in water (100 milliliters) was prepared at room temperature (about 23 degrees Celsius) in a jacketed flask. OXONE™ (6.15 grams, 10 mmol) was added to the solution. After stirring for about 2 hours a solid product precipitated from solution. The solid product was filtered from the solution and washed with water, yielding essentially pure 3,3′-diamino-,4′-azoxyfurazan (“DMFA”). The yield was 72%.

### Example 2

An aqueous solution of DAF (1.02 grams, 10.2 mmol) and sodium bicarbonate (5.042 grams, 60 mmol) in water (100 milliliters) was prepared at room temperature (about 23 degrees Celsius) in a jacketed flask. OXONE™ (12.32 grams, 20 mmol) was added to the solution. After stirring for 4 hours, a solid product precipitated from solution. The solid product was filtered from the solution and washed with water, yielding essentially pure DAAF. The yield was 70%.

### Example 3

An aqueous solution of DAF (0.102 grams, 1.02 mmol) and sodium carbonate (0.308 grams, 2.9 mmol) in water (5 milliliters) was prepared at room temperature (about 23 degrees Celsius) in a flask. OXONE™ (1.23 grams, 2 mmol) was added to the solution. After stirring for about 4 hours, a solid product precipitated from the solution. The solid product was filtered from the solution and washed with water, yielding essentially pure DAAF. The yield was 55%.

### Example 4

In this EXAMPLE, the procedure followed was that of EXAMPLE 1 with the exception that after stirring for about two hours, additional sodium bicarbonate (2.52 grams, 30 mmol) and additional OXONE™ (6.15 grams, 10 mmol) was added and stirring was continued for about another 2 hours. A solid product precipitated from the solution. The solid product was filtered from the solution and washed with water, yielding essentially pure DAAF. The yield was 82%.

### Example 5

In this EXAMPLE, the procedure was that of EXAMPLE 4 with the exception that after the second addition of sodium bicarbonate and OXONE™ followed by stirring for two hours, additional sodium bicarbonate (2.52 grams, 30 mmol) and OXONE™ (6.15 grams, 10 mmol) were added. Thus, the total reaction time for this EXAMPLE was 6 hours, the total amount of OXONE™ was 18.45 grams (30 mmol), and the total amount of sodium bicarbonate was 7.56 grams (90 mmol). The product was essentially pure DAAF. The yield was 78%.

### Example 6

In this EXAMPLE, the procedure was that of EXAMPLE 1 with the exception that sodium bicarbonate was not added to the solution. The pH was measured after adding the OXONE™ and was determined to be pH=2. A DSC analysis showed that the product was DAAF but not essentially pure DAAF because it also contained impurities. The yield was 66%.

### Example 7

In this EXAMPLE, the procedure was that of EXAMPLE 1 with the exception that sodium acetate (2.46 grams, 30 mmol) was used instead of sodium bicarbonate. After adding the OXONE™, the pH was checked with pH paper and found to be pH=4. A DSC analysis showed that the DAAF-containing product contained impurities but in lesser amounts than those found in EXAMPLE 5. The yield was 77%.

### Example 8

In this EXAMPLE, the procedure followed was that of EXAMPLE 1 with the exception that the temperature of the solution was about 5 degrees Celsius instead of about 23 degrees Celsius. The product was essentially pure DMF. The yield was 66%.

### Example 9

In this EXAMPLE, the procedure followed was that of EXAMPLE 8 with the exception that (i) twice as much sodium bicarbonate and twice as much OXONE™ were used, and (ii) stirring was continued for 6 hours instead of 2 hours. The product was essentially pure DMF. The yield was 72%.

### Example 10

In this EXAMPLE, the procedure followed was that of EXAMPLE 8 with the exception that after the solution was stirred for 6 hours, additional sodium bicarbonate (2.52 grams, 30 mmol) and additional OXONE™ (6.15 grams, 10 mmol) were added and stirring was continued for another 17 hours. The product was essentially pure DAAF. The yield was 82%.

### Example 11

An aqueous solution of 3,4-diaminofurazan (“DAF”) (5.06 grams, 50 mmol) and sodium bicarbonate (12.62 grams, 150 mmol) in water (500 milliliters) was prepared at room temperature (about 23 degrees Celsius) in a jacketed flask. OXONE™ (30.65 grams, 50 mmol) was added to the solution. After stirring for about 2 hours, additional sodium bicarbonate (12.61 grams, 150 mmol) and additional OXONE™ (30.75 grams, 50 mmol) were added and stirring was continued for about another 2 hours. A solid product precipitated from the solution. The solid product was filtered from the solution and washed with water. The product was essentially pure DAAF. The yield was 81%. The mean particle size of the DAAF was determined to be 28 micrometers (“μm”).

### Example 12

In this EXAMPLE, the procedure followed was that of EXAMPLE 11 with the exception that the amount of water was decreased from 500 milliliters to 300 milliliters. The product was essentially pure DAAF. The yield was 80%.

### Example 13

An aqueous solution of DAF (15.065 grams, 0.15 moles) and sodium bicarbonate (37.81 grams, 0.45 moles) in water (1.5 Liters) was prepared at room temperature (about 22 degrees Celsius) in a jacketed flask. OXONE™ (92.28 grams, 0.15 moles) was added to the solution. After stirring for about 2 hours, additional sodium bicarbonate (37.85 grams, 0.45 moles) and additional OXONE™ (92.285 grams, 0.15 moles) were added and stirring was continued for about another 2 hours. A solid product precipitated from the solution. The solid product was filtered from the solution and washed with water, yielding essentially pure. DAAF. The yield was 83%.

### Example 14

An aqueous solution of DAF (50.09 grams, 0.50 moles) and sodium bicarbonate (126.06 grams, 1.5 moles) in water (about 3 Liters) was prepared at room temperature (about 22 degrees Celsius) in a 5-Liter jacketed flask (pH=7.91). OXONE™ (307.55 grams, 0.5 moles) was added to the solution and the pH was measured at pH=6.98. After stirring for about 2 hours, the pH was measured at pH=6.43. Additional sodium bicarbonate (126.05 grams, 1.5 moles) was added resulting in a pH=7.39. Additional OXONE™ (307.58 grams, 0.5 moles) was added, resulting in a pH of 7.22. Stirring was continued for about another 2 hours, during which time a solid product precipitated from the solution. The pH after 2 hours was 6.8. The solid product was filtered from the solution and washed with water, yielding 44.7 grams (89% yield) of essentially pure DMF.

### Example 15

An aqueous solution of DAF (150.0 grams, 1.5 moles) and sodium bicarbonate (378.2 grams, 4.5 moles) in water (about 9 Liters) was prepared at room temperature (about 22 degrees Celsius) in a 22-Liter non-jacketed flask. OXONE™ (922.5 grams, 1.5 moles) was added in three portions of about 300 grams each to the solution. After stirring for about 2 hours the pH was measured at pH=6.32. Additional sodium bicarbonate (378.0 grams, 4.5 moles) was added resulting in a pH=7.36. Additional OXONE™ (922.6 grams, 1.5 moles) was added, resulting in a pH=6.98. Stirring was continued for about another 2 hours, after which the pH was measured at pH=6.78. The solid product that precipitated from the solution was filtered and washed with water, yielding 134.3 grams (89.55%) of essentially pure DMF. The mean particle size of the DAAF was determined to be 41 μm.

### Example 16

In this EXAMPLE, the procedure of EXAMPLE 1 was followed with the exception that the temperature of the solution was 40 degrees Celsius instead of 23 degrees Celsius, and the solution was stirred for 30 minutes. The product was essentially pure DMF. The yield was 45%.

### Example 17

In this EXAMPLE, the procedure of EXAMPLE 16 was followed with the exception that the solution was stirred for about 2 hours instead of 30 minutes. The yield was 52% of essentially pure DAAF.

### Example 18

A solution of DAF (0.102 grams, 1.02 mmol) and meta-chloroperoxybenzoic acid (80%, 0.650 grams, 3.00 mmol) in acetonitrile (5 milliliters) was prepared at room temperature (about 23 degrees Celsius) in a flask. After stirring for about 5 days, the solid precipitate that formed was filtered from the solution and washed with acetonitrile. The product was essentially pure DMF. The yield was 75%.

### Example 19

An aqueous solution of DAF (0.102 grams, 1 mmol) and magnesium monoperphthalate (0.741 grams, 1.5 mmol) in water (10 milliliters) was prepared at room temperature (about 23 degrees Celsius) in a flask. After stirring for 18 hours, the solid precipitate that formed was filtered from the solution and washed with water. The product contained DAAF and impurities. The yield was 65%.

### Example 20

An aqueous solution of DAF (0.102 grams, 1.02 mmol) in a buffer having a pH of 7 and including sodium dihydrogen phosphate (“NaH₂PO₄”) and disodium hydrogen phosphate (“Na₂HPO₄”) at room temperature (about 23 degrees Celsius) was prepared. OXONE™ (1.23 grams, 2 mmol) was added to the solution. After stirring the resulting solution for 4 hours, a solid precipitated from the solution. The solid was filtered from the solution and washed with water. The solid product was essentially pure DAAF. The yield was 60%.

### Example 21

DAF (0.102 grams, 1.02 mmol) was added to acetic acid (10 ml) at room temperature (about 23 degrees Celsius) in a flask. Hydrogen peroxide (30%, 0.25 ml) was then added. After stirring for about 1 day, a solid precipitated from the solution. The solid was filtered from the solution and washed with cold acetic acid and dried. The solid product was essentially pure DAAF. The yield was 62%.

### Example 22

DAF (0.102 grams, 1.02 mmol) was added to an aqueous solution of sodium hypochlorite (12%, 10 ml) at room temperature (about 23 degrees Celsius). After stirring for about 8 hours, a solid precipitated from the solution. The solid was filtered from the solution and washed with cold water and dried. Analysis of the solid product showed that the product was not DAAF, but instead was 3,3′-4,4′-azofurazan (“DAAzF”). The yield was 50%.

### Example 23

Preparation of 3,3′-diamino-4,4′-azofurazan (“DAAzF”). DAF (0.500 grams, 5.00 mmol) was added to an aqueous solution of sodium bicarbonate (0.84 g, 10 mmol) and 10 ml of water at room temperature (about 23 degrees Celsius). A solution of 5% NaOCl (i.e. household bleach, 15 ml) was then added slowly over 15 minutes to the DAF/sodium bicarbonate mixture. The reaction was then stirred an additional 15 minutes. The orange solid was filtered from the solution and washed with cold water and dried. Analysis of the solid product showed that the product was not DAAF, but instead was 3,3′-diamino-4,4′-azofurazan. The yield was 90%.

### Example 24

DAF (0.102 grams, 1.02 mmol) was added to sodium percarbonate (0.314 g, 1 mmol) in water (10 ml) at room temperature (about 23 degrees Celsius) in a flask. After stirring for about 8 hours, no reaction was observed.

### Example 25

DAF (0.102 grams, 1.02 mmol) was added to sodium perborate (1 mmol) in water (10 ml) at room temperature (about 23 degrees Celsius) in a flask. After stirring for about 8 hours, no reaction was observed.

### Example 26

A number of pressed pellets of essentially pure DAAF of EXAMPLE 14 were prepared. The densities of the pellets were measured from 1.66 grams per cubic centimeter (“g/cc”) to 1.73 g/cc per individual pellet. The pellets were then subjected to detonation velocity experiments. The detonation velocities were measured using pins and/or switches and were calculated using streak camera images. On larger experiments (½″ rate sticks) a CJ pressure (“P_{cj}”) was measured from a plate dent. At a density of 1.66 g/cc the P_{cj} was 291 kbar. At a density of 1.69 g/cc, the Pcj was 306 kbar. A ½″ pressed pellet stack of essentially pure DAAF having a density of 1.69 grams per cubic centimeter and a booster of PBX 9407 had a detonation velocity of 8 kilometers per second. TABLE 1 summarizes the results.

TABLE 1

| | | | Diameter in |
|---|---|---|---|
| | Average Density | Detonation | millimeters |
| Sample number | (g/cc) | Velocity (km/s) | (“mm”) |
| DAAF-MA-50.1 | 1.66 | 7.89 | 12.7 |
| DAAF-MA-50.1 | 1.69 | 8.0 | 6.35 |
| DAAF-MA-50.1 | 1.71 | 7.91 | 5.0 |
| DAAF-MA-50.1 | 1.71 | 7.90 | 3.81 |
| DAAF-MA-50.1 | 1.73 | 7.94 | 3.0 |

As can be seen from TABLE 1, as the density of the pellet increased, the detonation velocity also increases, with the exception of the 6.35 mm stack which showed an unusually fast detonation. As the diameter of the pellet decreases, the detonation velocity decreases only slightly. Thus, the detonation velocity of the DAAF is not strongly influenced by the diameter, which is an unusual feature for an explosive.

The foregoing description of the invention has been presented for purposes of illustration and description and is not intended to be exhaustive or to limit the invention to the precise form disclosed, and obviously many modifications and variations are possible in light of the above teaching. For example, sodium bicarbonate and sodium carbonate are only exemplary of materials that form a buffer by reacting with acid generated in the reaction, and that other materials and other buffers could also perform this function without significantly affecting the purity of the DAAF product.

The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

1. A method for preparing 3,3′-diamino-4,4′-azoxyfurazan (“DAAF”), comprising reacting an aqueous solution comprising 3,4-diaminofurazan (“DAF”) with a material of the formula 2 KHSO₅.KHSO₄.K₂SO₄ in the presence of a chemical buffer or a material that produces a chemical buffer during the reaction of the DAF with the material of the formula 2 KHSO₅.KHSO₄.K₂SO₄, wherein DAAF precipitates from the aqueous solution.

2. The method of claim 1, wherein the buffer or the material that produces a chemical buffer results in a pH of the aqueous solution of from about pH 6.0 to about pH 8.0.

3. The method of claim 1, wherein the material that produces the chemical buffer comprises at least one of sodium bicarbonate and sodium carbonate.

4. The method of claim 1, wherein the chemical buffer comprises NaH₂PO₄.

5. The method of claim 1, further comprising separating the precipitated DAAF from the aqueous solution.

6. The method of claim 5, further comprising washing the DAAF.

7. A method for preparing a pressed article of essentially pure 3,3′-diamino-4,4′-azoxyfurazan (“DAAF”), comprising reacting an aqueous solution comprising 3,4-diaminofurazan (“DAF”) with a material of the formula 2KHSO₅.KHSO₄.K₂SO₄ in the presence of a chemical buffer or a material that produces a chemical buffer during the reaction of the DAF with the material of the formula 2 KHSO₅.KHSO₄.K₂SO₄, wherein DAAF precipitates from the aqueous solution, washing the DAAF, and thereafter without recrystallizing the DAAF, forming a pressed article from the DAAF, thereby forming a pressed article of essentially pure DAAF.

8. A pressed article of essentially pure DAAF having a density of 1.69 grams per cubic centimeter and a detonation velocity of 8 kilometers per second.

9. A method for preparing 3,3′-diamino-4,4′-azofurazan (“DAAzF”), comprising reacting an aqueous solution comprising 3,4-diaminofurazan (“DAF”) with sodium hypochlorite.

10. The method of claim 9, wherein the sodium hypochlorite comprises an aqueous solution of sodium hypochlorite.

11. The method of claim 10, wherein the reaction of 3,4-diaminofurazan and sodium hypochlorite is performed in the presence of a buffer.
